(19) United States
(12) Patent Application Publication
(10) Pub. No.: US 2025/0114383 A1
(43) Pub. Date: Apr. 10, 2025

(54) ADMINISTRATION OF A PSYCHEDELIC COMPOUND BY INTRAVENOUS INFUSION

(71) Applicant: RESET PHARMACEUTICALS, INC., New York, NY (US)

(72) Inventors: Harri DICKINSON, Cheadle (GB); Paul Alfred DICKINSON, Cheadle (GB); Nicola PARISI, Cheadle (GB)

(21) Appl. No.: 18/865,125

(22) PCT Filed: May 12, 2023
(86) PCT No.: PCT/US2023/022027

§ 371 Date: Nov. 12, 2024

Related U.S. Application Data
(60) Provisional application No. 63/364,632, filed on May 13, 2022.
(60) Provisional application No. 63/364,633, filed on May 13, 2022.
(60) Provisional application No. 63/364,634, filed on May 13, 2022.

(30) Foreign Application Priority Data
May 26, 2022 (GB) 2207765.5
May 26, 2022 (GB) 2207766.3
May 26, 2022 (GB) 2207767.1

(51) Int. Cl. A61K 31/675; A61K 9/00; A61K 31/4045; A61K 47/40

(52) U.S. Cl. CPC A61K 31/675; A61K 9/0019; A61K 31/4045; A61K 47/40

(57) ABSTRACT

The invention relates to a method of treating or preventing a disease or condition comprising administering a therapeutically effective amount of a psychedelic compound to a patient by intravenous infusion. Uses, kits, formulations and devices are also described.

### FIELD OF THE INVENTION

The present invention relates to methods of treating or preventing a disease or condition, comprising administering a psychedelic compound to a patient. Kits, formulations and devices are also described.

### BACKGROUND OF THE INVENTION

Psilocybin (4-phosphoryloxy-N,N-dimethyltryptamine) is a tryptamine serotoninergic psychedelic. The IUPAC name of psilocybin is [3-(2-dimethylaminoethyl)-1H-indol-4-yl]dihydrogen phosphate. The structure of psilocybin is shown below.

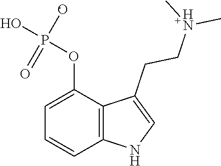

Psilocybin is metabolized in the body to psilocin (4-hydroxy-N,N-dimethyltryptamine), which exerts its effects primarily via 5HT2A agonism. The structure of psilocin is shown below.

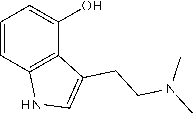

Psilocybin and psilocin are currently being investigated as a potential treatment for various psychological, neurological and central nervous system disorders (for example, demoralization, depression, anxiety and adjustment disorders) in a variety of clinical settings.

Psilocybin is typically dosed orally. Following oral dosing, the onset of action typically starts between 20-40 minutes, with maximum effect at 60-90 minutes and a duration of 4-8 hours. A faster onset of action and a shorter duration of the psychological effect than those reported above for oral administration would be desirable when psilocybin or psilocin are used as therapeutic agents. Furthermore, the bioavailability of psilocybin has been reported to be approximately 50%. An improvement of the bioavailability would allow a desirable reduction of the psilocybin dose needed to elicit its action.

Intravenous (IV) administration of psilocybin has been reported to have an onset of action of 1-2 minutes, and a duration of the psychological effects of 20 minutes. A slower onset of action and a longer duration of the psychological effects than those previously observed for IV administration need to be achieved for the IV administration of psilocybin or psilocin to be therapeutically useful, for example for treatment of psychiatric disorders. An improved control of the pharmacokinetic (PK) profile would allow optimization of the dose of psilocybin or psilocin and reduction of PK variability, improving the effectiveness of treatment and reducing adverse effects.

There is accordingly a need to develop new methods of administration of psilocybin and psilocin which allow improvements to the control of the pharmacokinetic profile to be obtained. Such new methods would provide improved treatments, for example when psilocybin or psilocin are used in the treatment of psychiatric disorders.

### SUMMARY OF THE INVENTION

It is a finding of the invention that an IV infusion dosage form of psilocybin or psilocin provides improved methods of treatment or prevention of diseases or conditions. In particular, controlling the infusion rate and dose administered for an IV infusion dosage form of psilocybin or psilocin allows a desirable onset of action, duration of psychological effects and pharmacokinetic (PK) profile to be achieved when the psilocybin or psilocin are administered to a patient.

The present invention accordingly provides a method of treating or preventing a disease or condition in a patient, the method comprising administering a therapeutically effective amount of a psychedelic compound to the patient by intravenous infusion, wherein the psychedelic compound is psilocybin or psilocin, or a pharmaceutically acceptable salt thereof.

The invention further provides a psychedelic compound for use in a method of treating or preventing a disease or condition in a patient, wherein: the psychedelic compound is psilocybin or psilocin, or a pharmaceutically acceptable salt thereof; and the method comprises administering a therapeutically effective amount of the psychedelic compound to the patient by intravenous infusion.

The invention also provides use of a psychedelic compound in the manufacture of a medicament for use in a method of treating or preventing of a disease or condition in a patient, wherein: the psychedelic compound is psilocybin or psilocin, or a pharmaceutically acceptable salt thereof; and the method comprises administering a therapeutically effective amount of the psychedelic compound to the patient by intravenous infusion.

The disease or condition may be selected from psychological, neurological and central nervous system disorders.

The invention further provides a formulation suitable for administration by intravenous infusion comprising a psychedelic compound which is psilocin or a pharmaceutically acceptable salt thereof, wherein the formulation is a solution and the formulation comprises psilocin at a concentration of at least about 200 mg/g.

Also provided by the invention is a kit comprising: one or more infusion bags, syringes, vials and/or ampoules comprising a formulation suitable for administration by intravenous infusion, which formulation comprises a psychedelic compound which is psilocybin or psilocin, or a pharmaceutically acceptable salt thereof; and instructions for use of the one or more infusion bags, syringes, vials and/or ampoules.

Also provided by the invention is a device which is configured to administer a psychedelic compound to a patient by intravenous infusion, wherein the psychedelic compound is psilocybin or psilocin, or a pharmaceutically acceptable salt thereof.

### BRIEF DESCRIPTION OF THE FIGURES

FIG. 1 shows a model schematic of a two-compartment IV bolus model.

FIG. 2 shows visualized plasma concentration data.

FIG. 3 shows a fitted two compartment IV PK model of psilocin. The line is a simulated PK profile and black dots are the IV data.

FIG. 4 shows a model schematic of a two-compartment model with one zero order infusion.

FIG. 5 shows a model schematic of a two-compartment model with two zero order infusions.

FIG. 6 shows human simulations of psilocin exposure after IV 90-minute infusion administration over a range of doses, where the plot is faceted by dose in mg.

FIG. 7 shows human simulations of psilocin exposure after two infusion rates over 7-10 mg total doses. The darker line shows simulated two infusion PK profile and the lighter line shows simulated 25 mg exposure after oral administration.

### DETAILED DESCRIPTION OF THE INVENTION

The method of the invention comprises administering a therapeutically effective amount of a psychedelic compound to the patient by intravenous infusion. In the present invention, the psychedelic compound is psilocybin or psilocin, or a pharmaceutically acceptable salt thereof. In one embodiment, the psychedelic compound is psilocybin or a pharmaceutically acceptable salt thereof. In one embodiment, the psychedelic compound is psilocin or a pharmaceutically acceptable salt thereof.

As used herein, the term “intravenous infusion” or “IV infusion” means administration of a substance directly into a subject's veins.

The psychedelic compound administered to the patient may be in any pharmaceutically acceptable form. Typically, the psychedelic compound is in administered to the patient in the form of a solution comprising the psychedelic compound.

The psychedelic compound may be administered to the patient by any suitable method for intravenous infusion. For example, the psychedelic compound may be administered to the patient using a syringe and a syringe driver. Alternatively, the psychedelic compound may be administered to the patient using an infusion bag under gravity. When an infusion bag is used, a clamp may additionally be utilised to assist in controlling the rate of administration of the psychedelic compound to the patient.

As used herein, a “syringe driver”, also known as a “syringe pump” or an “infusion pump”, is a device used to administer infusions, for example intravenous infusions. Syringe drivers allow the rate, total time, total dose etc. of administration of a medicament to a patient to be controlled.

The psychedelic compound may be administered to the patient for any suitable length of time. For example, the method may comprise administering the therapeutically effective amount of the psychedelic compound to a patient by intravenous infusion for a total time of from about 2 minutes to about 250 minutes, from about 5 minutes to about 200 minutes, from about 30 minutes to about 190 minutes, from about 45 minutes to about 180 minutes, from about 50 minutes to about 170 minutes, from about 60 minutes to 150 minutes, from about 70 minutes to about 130 minutes, or from about 80 minutes to about 110 minutes.

As used herein, the term “about” means any value that the skilled person would appreciate is a reasonable variation of the value that is referred to by the term “about”. Typically, “about” means ±10% or ±5%.

The method may comprise administering the therapeutically effective amount of the psychedelic compound to a patient by intravenous infusion for a total time of from, for example, from about 30 minutes to about 40 minutes, from about 40 minutes to about 50 minutes, from about 50 minutes to about 60 minutes, from about 70 minutes to about 80 minutes, from about 80 minutes to about 90 minutes, from about 90 minutes to about 100 minutes, from about 100 minutes to about 110 minutes, from about 110 minutes to about 120 minutes, from about 120 minutes to about 130 minutes, from about 130 minutes to about 140 minutes, from about 140 minutes to about 150 minutes, or from about 150 minutes to about 160 minutes.

The therapeutically effective amount of the psychedelic compound may be from about 0.5 mg to about 20 mg, from about 1 mg to about 15 mg, from about 3 mg to about 14 mg, from about 4 mg to about 13.5 mg, from about 5 mg to about 13 mg, from about 5.5 mg to about 12.5 mg, from about 6 mg to about 12 mg, from about 6.5 mg to about 11.8 mg, from about 7 mg to about 11.5 mg, from about 7.5 mg to about 11.3 mg, or from about 8 mg to about 11 mg. In one embodiment, the psychedelic compound is psilocybin and the therapeutically effective amount is from about 6 mg to about 12 mg or from about 8 mg to about 11 mg. In another embodiment, the psychedelic compound is psilocin and the therapeutically effective amount is from about 4.5 mg to about 8.5 mg or from about 6 mg to about 8 mg.

For example, the therapeutically effective amount of the psychedelic compound may be from about 1.5 mg to about 2.5 mg, from about 2.5 mg to about 3.5 mg, from about 3.5 mg to about 4.5 mg, from about 4.5 mg to about 5.5 mg, from about 5.5 mg to about 6.5 mg, from about 6.5 mg to about 7.5 mg, from about 7.5 mg to about 8.5 mg, from about 8.5 mg to about 9.5 mg, from about 9.5 mg to about 10.5 mg, from about 10.5 mg to about 11.5 mg, from about 11.5 mg to about 12.5 mg, from about 12.5 mg to about 13.5 mg, or from about 13.5 mg to about 14.5 mg.

The psychedelic compound may be administered to the patient in an amount of from about 1 mg to about 15 mg and for a time of from about 5 minutes to about 200 minutes, or in an amount of from about 3 mg to about 14 mg and for a time of from about 30 minutes to about 190 minutes, or in an amount of from about 5 mg to about 13 mg and for a time of from about 45 minutes to about 180 minutes, or in an amount of from about 6 mg to about 12 mg and for a time of from about 50 minutes to about 170 minutes. In one embodiment, the psychedelic compound is psilocybin and the psychedelic compound is administered to the patient in an amount of from about 5 mg to about 13 mg and for a time of from about 45 minutes to about 180 minutes. In another embodiment, the psychedelic compound is psilocin and the psychedelic compound is administered to the patient in an amount of from about 3.5 mg to about 9.5 mg and for a time of from about 45 minutes to about 180 minutes.

The psychedelic compound may be administered to the patient in an amount of from 1.5 mg to about 2.5 mg and for a total time of from about 40 minutes to about 50 minutes, from about 2.5 mg to about 3.5 mg and for a total time of from about 50 minutes to about 60 minutes, from about 3.5 mg to about 4.5 mg and for a total time of from about 60 minutes to about 70 minutes, from about 4.5 mg to about 5.5 mg and for a total time of from about 70 minutes to about 80 minutes, from about 5.5 mg to about 6.5 mg and for a total time of from about 80 minutes to about 90 minutes, from about 6.5 mg to about 7.5 mg and for a total time of from about 90 minutes to about 100 minutes, from about 7.5 mg to about 8.5 mg and for a total time of from about 110 minutes to about 120 minutes, from about 8.5 mg to about 9.5 mg and for a total time of from about 120 minutes to about 130 minutes, from about 9.5 mg to about 10.5 mg and for a total time of from about 130 minutes to about 140 minutes, from about 10.5 mg to about 11.5 mg and for a total time of from about 140 minutes to about 150 minutes, or from about 11.5 mg to about 12.5 mg and for a total time of from about 150 minutes to about 160 minutes.

The psychedelic compound may be administered to the patient at any suitable infusion rate. The infusion rate may be fixed throughout the administration of the psychedelic compound to the patient, or the infusion rate may vary during the administration. When the infusion rate varies, the term “infusion rate” refers to the average rate of infusion over the administration.

For example, the method may comprise administering the therapeutically effective amount of the psychedelic compound to a patient by intravenous infusion at an infusion rate of from about 0.005 mg/min to about 0.5 mg/min, from about 0.008 mg/min to about 0.4 mg/min, from about 0.01 mg/min to about 0.3 mg/min, from about 0.03 mg/min to about 0.25 mg/min, from about 0.05 mg/min to about 0.2 mg/min, from about 0.07 mg/min to about 0.15 mg/min, or from about 0.09 mg/min to about 0.13 mg/min. In one embodiment, the psychedelic compound is psilocybin and the infusion rate is from about 0.07 mg/min to about 0.15 mg/min or from about 0.09 mg/min to about 0.13 mg/min. In another embodiment, the psychedelic compound is psilocin and the infusion rate is from about 0.05 mg/min to about 0.10 mg/min or from about 0.06 mg/min to about 0.09 mg/min.

Thus, it is typical to administer the psychedelic compound to the patient at an infusion rate of from about 0.01 mg/min to about 0.02 mg/min, from about 0.02 mg/min to about 0.03 mg/min, from about 0.03 mg/min to about 0.04 mg/min, from about 0.04 mg/min to about 0.05 mg/min, from about 0.05 mg/min to about 0.06 mg/min, from about 0.06 mg/min to about 0.07 mg/min, from about 0.07 mg/min to about 0.08 mg/min, from about 0.08 mg/min to about 0.09 mg/min, from about 0.09 mg/min to about 0.1 mg/min, from about 0.1 mg/min to about 0.11 mg/min, from about 0.11 mg/min to about 0.12 mg/min, from about 0.12 mg/min to about 0.13 mg/min, from about 0.13 mg/min to about 0.14 mg/min, from about 0.14 mg/min to about 0.15 mg/min, from about 0.15 mg/min to about 0.16 mg/min, from about 0.16 mg/min to about 0.17 mg/min, from about 0.17 mg/min to about 0.18 mg/min, from about 0.18 mg/min to about 0.19 mg/min, or from about 0.19 mg/min to about 0.2 mg/min.

As discussed above, an IV infusion dosage form of the psychedelic compound allows a desirable onset of action, and duration of psychological effects to be obtained. In particular, the method of the present invention allows a tuneable PK profile to be obtained by controlling the dose and/or rate of infusion of the psychedelic compound to a patient.

As used herein, the term “duration of psychological effects” refers to the total time that a therapeutic effect of the compound is observed for. This may be determined by measuring psilocin levels in the patient's blood plasma. In that case, psychological effects may be observed when the blood plasma concentration of psilocin is above about 5 ng/mL. Alternatively, the onset and end of the psychological effects may be based on observation by a medical professional, or they may be self-reported by the patient.

A finding of the present invention is that an advantageous maximum blood plasma concentration (Cₘₐₓ) of psilocin (i.e. the psychedelic compound itself when psilocin is administered, or the active metabolite when psilocybin is administered) may be obtained by administering the psychedelic compound to a patient by intravenous infusion as discussed herein. Such advantageous Cₘₐₓ value of psilocin provides a desirable therapeutic effect, thus providing effective treatment or prevention of the diseases, disorders and conditions described herein, without causing an excessive duration of the psychological effect in the patient.

For example, administering the therapeutically effective amount of the psychedelic compound to the patient by intravenous infusion may achieve a maximum blood plasma concentration (Cₘₐₓ) of psilocin in the patient which is at least about 3 ng/mL, at least about 4 ng/mL, at least about 5 ng/mL, at least about 6 ng/mL, at least about 7 ng/mL, at least about 8 ng/mL, at least about 9 ng/mL, at least about 10 ng/mL, at least about 11 ng/mL, at least about 12 ng/mL, at least about 13 ng/mL, at least about 14 ng/mL, or at least about 15 ng/mL.

For example, administering the therapeutically effective amount of the psychedelic compound to the patient by intravenous infusion may achieve a maximum blood plasma concentration (Cₘₐₓ) of psilocin in the patient which is no greater than about 20 ng/mL, no greater than about 19 ng/mL, no greater than about 18 ng/mL, no greater than about 17 ng/mL, no greater than about 16 ng/mL, no greater than about 15 ng/mL, no greater than about 14 ng/mL, no greater than about 13 ng/mL, no greater than about 12 ng/mL, no greater than about 11 ng/mL, or no greater than about 10 ng/mL.

Thus, administering the therapeutically effective amount of the psychedelic compound to the patient by intravenous infusion may achieve a maximum blood plasma concentration (Cₘₐₓ) of psilocin in the patient which is, for example, from about 6 ng/mL to about 7 ng/mL, from about 7 ng/mL to about 8 ng/mL, from about 8 ng/mL to about 9 ng/mL, from about 9 ng/mL to about 10 ng/mL, from about 10 ng/mL to about 11 ng/mL, from about 11 ng/mL to about 12 ng/mL, from about 12 ng/mL to about 13 ng/mL, from about 13 ng/mL to about 14 ng/mL, from about 14 ng/mL to about 15 ng/mL, from about 15 ng/mL to about 16 ng/mL, from about 16 ng/mL to about 17 ng/mL, from about 17 ng/mL to about 18 ng/mL, from about 18 ng/mL to about 19 ng/mL, or from about 19 ng/mL to about 20 ng/mL.

In general, during the method of the invention it is desirable to achieve a blood plasma concentration of psilocin that is between about 5 ng/mL and about 10 ng/mL for an extended period of time over the course of the treatment. This range is preferred in particular when the method is used in the treatment of prevention of psychological, neurological and central nervous system disorders.

As discussed above, it is desirable for the method of the present invention to result in a faster onset of action and a shorter duration of the psychological effect than those reported for oral administration when psilocybin or psilocin are used as therapeutic agents.

It is generally desirable for the method of the invention to achieve onset of therapeutic action relatively quickly after initiating administration of the intravenous infusion. Thus, administering the therapeutically effective amount of the psychedelic compound to the patient by intravenous infusion may achieve onset of therapeutic action in a time from onset of intravenous infusion of less than about 90 minutes, or less than about 80 minutes, or less than about 70 minutes, or less than about 60 minutes, or less than about 50 minutes, or less than about 40 minutes, or less than about 30 minutes, or less than about 20 minutes, or less than about 10 minutes, or less than about 5 minutes.

As used herein, the term “onset of therapeutic action” refers to the point during administration of the psychedelic compound where the therapeutic effect of the compound is first observed. This may be determined by measuring psilocin levels in the patient's blood plasma. In that case, the blood plasma concentration may be around about 5 ng/mL at the point of onset of therapeutic action. Alternatively, the onset of therapeutic action may be based on observation by a medical professional, or it may be self-reported by the patient during the administration of the compound.

It is also generally desirable for the onset of therapeutic action to not be too rapid after initiating administration of the intravenous infusion. Thus, administering the therapeutically effective amount of the psychedelic compound to the patient by intravenous infusion may achieve onset of therapeutic action in a time from onset of intravenous infusion of greater than about 1 minutes, or greater than about 2 minutes, or greater than about 3 minutes, or greater than about 4 minutes, or greater than about 5 minutes, or greater than about 6 minutes, or greater than about 7 minutes.

Thus, onset of therapeutic action is typically from about 2 minutes to about 60 minutes, or from about 3 minutes to about 50 minutes, or from about 4 minutes to about 40 minutes, or from about 5 minutes to about 30 minutes, or from about 6 minutes to about 20 minutes after initiating administration of the intravenous infusion.

Administering the therapeutically effective amount of the psychedelic compound to the patient by intravenous infusion may achieve a blood plasma concentration of psilocin of greater than or equal to about 5 ng/mL in a time from onset of intravenous infusion of from about 1 to about 60 minutes, or from about 2 to about 50 minutes, or from about 3 to about 40 minutes, or from about 4 to about 30 minutes, or from about 5 to about 20 minutes. Typically, administering the therapeutically effective amount of the psychedelic compound to the patient by intravenous infusion achieves a blood plasma concentration of psilocin of greater than or equal to about 5 ng/mL in a time from onset of intravenous infusion of from about 6 to about 15 minutes.

The method of the invention may achieve a blood plasma concentration of psilocin of greater than about 5 ng/mL for a relatively short period of time, in particular to provide a shorter duration of the psychological effect than can be obtained by oral administration of psilocybin or psilocin. This provides improvements when the method of the invention is used, for example, to treat disorders such as psychological, neurological and central nervous system disorders. In particular, the relatively short duration of psychological effects provided by the invention may reduce the duration of time that a patient must be supervised and/or monitored following therapy with a psychedelic agent, such as psilocybin or psilocin.

Thus, administering the therapeutically effective amount of the psychedelic compound to the patient by intravenous infusion may result in the blood plasma concentration of psilocin in the patient being greater than or equal to about 5 ng/mL for a time of less than about 320 minutes, or less than about 300 minutes, or less than about 280 minutes, or less than about 260 minutes, or less than about 240 minutes, or less than about 220 minutes, or less than about 200 minutes, or less than about 180 minutes, or less than about 160 minutes, or less than about 140 minutes, or less than about 120 minutes, or less than about 100 minutes, or less than about 80 minutes, or less than about 60 minutes, or less than about 40 minutes.

Administering the therapeutically effective amount of the psychedelic compound to the patient by intravenous infusion may achieve a blood plasma concentration of psilocin in the patent of greater than or equal to about 5 ng/mL for a time of from about 80 to about 320 minutes, or from about 100 to about 300 minutes, for from about 120 to about 280 minutes, or from about 140 to about 260 minutes. Typically, administering the therapeutically effective amount of the psychedelic compound to the patient by intravenous infusion results in the blood plasma concentration of psilocin in the patient being greater than or equal to about 5 ng/mL for a time of from about 180 to about 240 minutes.

When the method of the invention achieves a blood plasma concentration of psilocin of greater than or equal to about 5 ng/mL in a time from onset of intravenous infusion of less than about 90 minutes, it may take less than about 95 minutes for the blood plasma concentration of psilocin to return to about 5 ng/mL or less. When the method of the invention achieves a blood plasma concentration of psilocin of greater than or equal to about 5 ng/mL in a time from onset of intravenous infusion of less than about 55 minutes, it may take less than about 100 minutes for the blood plasma concentration of psilocin to return to about 5 ng/mL or less. When the method of the invention achieves a blood plasma concentration of psilocin of greater than or equal to about 5 ng/mL in a time from onset of intravenous infusion of less than about 35 minutes, it may take less than about 125 minutes for the blood plasma concentration of psilocin to return to about 5 ng/mL or less. When the method of the invention achieves a blood plasma concentration of psilocin of greater than or equal to about 5 ng/mL in a time from onset of intravenous infusion of less than about 25 minutes, it may take less than about 150 minutes for the blood plasma concentration of psilocin to return to about 5 ng/mL or less. When the method of the invention achieves a blood plasma concentration of psilocin of greater than or equal to about 5 ng/mL in a time from onset of intravenous infusion of less than about 20 minutes, it may take less than about 170 minutes for the blood plasma concentration of psilocin to return to about 5 ng/mL or less. When the method of the invention achieves a blood plasma concentration of psilocin of greater than or equal to about 5 ng/mL in a time from onset of intravenous infusion of less than about 15 minutes, it may take less than about 190 minutes for the blood plasma concentration of psilocin to return to about 5 ng/mL or less. When the method of the invention achieves a blood plasma concentration of psilocin of greater than or equal to about 5 ng/mL in a time from onset of intravenous infusion of less than about 10 minutes, it may take less than about 195 minutes for the blood plasma concentration of psilocin to return to about 5 ng/mL or less. When the method of the invention achieves a blood plasma concentration of psilocin of greater than or equal to about 5 ng/mL in a time from onset of intravenous infusion of less than about 8 minutes, it may take less than about 210 minutes for the blood plasma concentration of psilocin to return to about 5 ng/mL or less. When the method of the invention achieves a blood plasma concentration of psilocin of greater than or equal to about 5 ng/mL in a time from onset of intravenous infusion of less than about 6.5 minutes, it may take less than about 220 minutes for the blood plasma concentration of psilocin to return to about 5 ng/mL or less. When the method of the invention achieves a blood plasma concentration of psilocin of greater than or equal to about 5 ng/mL in a time from onset of intravenous infusion of less than about 5.5 minutes, it may take less than about 235 minutes for the blood plasma concentration of psilocin to return to about 5 ng/mL or less.

### Administration of Two or More Doses of the Psychedelic Compound

It is a finding of the invention that an improved PK profile of psilocin can be obtained by administering two or more doses of the psychedelic compound by intravenous infusion, wherein the two or more doses are administered at distinct infusion rates and/or infusion times. Thus, administering the therapeutically effective amount of the psychedelic compound to the patient by intravenous infusion may comprise administering a first dose of said psychedelic compound at a first infusion rate for a first infusion time; and a second dose of said psychedelic compound at a second infusion rate for a second infusion time; wherein the first and second infusion rates are different.

The two or more doses of the psychedelic compound may be administered to the patient, for example, using a single syringe driver. In this case, the rate of infusion of the psychedelic compound by the syringe driver changes over time, to provide at least two distinct infusion rates.

Alternatively, the two or more doses of the psychedelic compound may be administered to the patient, for example, using two or more syringe drivers. In this case, at least two of the syringe drivers administer the psychedelic compound at distinct infusion rates. Each syringe driver may administer the psychedelic compound at a fixed infusion rate, or one or more of the syringe drivers may administer the psychedelic compound at a variable infusion rate.

The first infusion rate may be at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, at least about 150%, at least about 160%, at least about 170%, at least about 180%, at least about 190%, or at least about 200% greater than the second infusion rate.

The first infusion rate may be from about 0.01 mg/min to about 0.5 mg/min, from about 0.05 mg/min to about 0.3 mg/min, from about 0.08 mg/min to about 0.25 mg/min, or from about 0.1 mg/min to about 0.2 mg/min of the psychedelic compound. In one embodiment, the psychedelic compound is psilocybin and the first infusion rate is from about 0.1 mg/min to about 0.2 mg/min. In another embodiment, the psychedelic compound is psilocin and the first infusion rate is from about 0.07 mg/min to about 0.15 mg/min.

The second infusion rate may be from about 0.01 mg/min to about 0.15 mg/min, from about 0.02 mg/min to about 0.125 mg/min, from about 0.03 mg/min to about 0.1 mg/min, or from about 0.04 mg/min to about 0.08 mg/min of the psychedelic compound. In one embodiment, the psychedelic compound is psilocybin and the second infusion rate is from about 0.04 mg/min to about 0.8 mg/min. In another embodiment, the psychedelic compound is psilocin and the second infusion rate is from about 0.03 mg/min to about 0.6 mg/min.

The first infusion may be administered to the patient for a time (i.e. a first infusion time) of from about 1 minute to about 40 minutes, from about 5 minutes to about 30 minutes, from about 8 minutes to about 25 minutes, from about 10 minutes to about 23 minutes, from about 12 minutes to about 22 minutes, from about 14 minutes to about 21 minutes, or from about 15 minutes to about 20 minutes. Alternatively, the first infusion may be administered to the patient for a time (i.e. a first infusion time) of from about 8 to about 12 minutes.

The second infusion may be administered to the patient for a time (i.e. a second infusion time) of from about 45 minutes to about 200 minutes, from about 60 minutes to about 150 minutes, from about 75 minutes to about 145 minutes, from about 90 minutes to about 135 minutes, or from about 95 minutes to about 130 minutes.

The first dose is typically less than or equal to about 50%, less than or equal to about 45%, less than or equal to about 40%, less than or equal to about 35%, less than or equal to about 30%, less than or equal to about 25%, less than or equal to about 20%, less than or equal to about 15%, or less than or equal to 10% of the second dose.

The total dose of the psychedelic compound administered may be from about 6 mg to about 11 mg, with from about 5% to about 35% of the total dose being administered as the first dose. Alternatively, the total dose of the psychedelic compound administered may be from about 7 mg to about 10 mg, with from about 20% to about 35% of the total dose being administered as the first dose. Alternatively, the total dose of the psychedelic compound administered may be from about 7 mg to about 9 mg, with from about 25% to about 35% of the total dose being administered as the first dose. Alternatively, the total dose of the psychedelic compound administered may be from about 9 mg to about 11 mg, with from about 8% to about 12% of the total dose being administered as the first dose.

In one embodiment, the psychedelic compound is psilocybin and the total dose administered is from about 6 mg to about 11 mg, with from about 5% to about 35% of the total dose being administered as the first dose. In another embodiment, the psychedelic compound is psilocin and the total dose administered is from about 4 mg to about 8 mg, with from about 5% to about 35% of the total dose being administered as the first dose.

The first dose of the psychedelic compound may be from about from about 0.1 mg to about 0.3 mg and the first dose may be less than or equal to about 50%, less than or equal to about 45%, less than or equal to about 40%, less than or equal to 35%, less than or equal to about 30%, less than or equal to about 25%, less than or equal to about 20%, less than or equal to about 15%, or less than or equal to about 10% of the second dose. The first dose of the psychedelic compound may be from about 0.3 mg to about 0.5 mg and the first dose may be less than or equal to about 50%, less than or equal to about 45%, less than or equal to about 40%, less than or equal to 35%, less than or equal to about 30%, less than or equal to about 25%, less than or equal to about 20%, less than or equal to about 15%, or less than or equal to about 10% of the second dose. The first dose of the psychedelic compound may be from about 0.5 mg to about 0.7 mg and the first dose may be less than or equal to about 50%, less than or equal to about 45%, less than or equal to about 40%, less than or equal to 35%, less than or equal to about 30%, less than or equal to about 25%, less than or equal to about 20%, less than or equal to about 15%, or less than or equal to about 10% of the second dose. The first dose of the psychedelic compound may be from about 0.7 mg to about 0.9 mg and the first dose may be less than or equal to about 50%, less than or equal to about 45%, less than or equal to about 40%, less than or equal to 35%, less than or equal to about 30%, less than or equal to about 25%, less than or equal to about 20%, less than or equal to about 15%, or less than or equal to about 10% of the second dose. The first dose of the psychedelic compound may be from about 0.9 mg to about 1.1 mg and the first dose may be less than or equal to about 50%, less than or equal to about 45%, less than or equal to about 40%, less than or equal to 35%, less than or equal to about 30%, less than or equal to about 25%, less than or equal to about 20%, less than or equal to about 15%, or less than or equal to about 10% of the second dose. The first dose of the psychedelic compound may be from about 1.1 mg to about 1.3 mg and the first dose may be less than or equal to about 50%, less than or equal to about 45%, less than or equal to about 40%, less than or equal to 35%, less than or equal to about 30%, less than or equal to about 25%, less than or equal to about 20%, less than or equal to about 15%, or less than or equal to about 10% of the second dose. The first dose of the psychedelic compound may be from about 1.3 mg to about 1.5 mg and the first dose may be less than or equal to about 50%, less than or equal to about 45%, less than or equal to about 40%, less than or equal to 35%, less than or equal to about 30%, less than or equal to about 25%, less than or equal to about 20%, less than or equal to about 15%, or less than or equal to about 10% of the second dose. The first dose of the psychedelic compound may be from about 1.5 mg to about 1.7 mg and the first dose may be less than or equal to about 50%, less than or equal to about 45%, less than or equal to about 40%, less than or equal to 35%, less than or equal to about 30%, less than or equal to about 25%, less than or equal to about 20%, less than or equal to about 15%, or less than or equal to about 10% of the second dose. The first dose of the psychedelic compound may be from about 1.7 mg to about 1.9 mg and the first dose may be less than or equal to about 50%, less than or equal to about 45%, less than or equal to about 40%, less than or equal to 35%, less than or equal to about 30%, less than or equal to about 25%, less than or equal to about 20%, less than or equal to about 15%, or less than or equal to about 10% of the second dose.

The first dose is typically administered before the second dose; and administration of the second dose begins upon completion of administration of the first dose.

The method may further comprise administering a n^{th} dose of said psychedelic compound at a n^{th} infusion rate for a n^{th} infusion time, wherein n is 3, 4, 5, 6, 7, 8, 9, or 10. For example, the method may further comprise administering a third dose of said psychedelic compound at a third infusion rate for a third infusion time.

While the psychedelic compound may be administered by intravenous infusion at a number of fixed, discrete infusion rates, the psychedelic compound may also be administered by intravenous infusion where the rate of change of infusion rate is non-zero, i.e. where the rate of infusion varies over time such that there are not a small number of fixed, discrete infusion rates over the course of the administration of the psychedelic compound. In this case, an initial amount of the psychedelic compound may be administered to the patient, and the rate of infusion may then be increased or decreased to maintain the psychological effect of the psychedelic compound throughout the treatment. For example, a medical professional may manually increase or decrease the rate of administration of the psychedelic compound depending on the response of the patient to the treatment. Alternatively, the blood plasma concentration of psilocin in the patient may be monitored and the rate of intravenous infusion automatically adjusted to maintain a desirable blood plasma concentration of psilocin throughout the treatment. The psychedelic compound may for example be administered by a syringe driver which is programmed to change the rate of infusion over the course of the administration of the psychedelic compound.

In this embodiment, the average rate of intravenous infusion may decrease over the course of the administration, i.e. the average rate of the intravenous infusion may be higher during the first half of the administration than during the second half of the administration.

For example, the method may comprise administering, from the start of administration, from about 5% to about 70% of the total dose of the psychedelic compound to the patient within about 1 minute to about 40 minutes, or from about 10% to about 65% of the total dose of the psychedelic compound to the patient within about 5 minutes to about 30 minutes, or from about 15% to about 60% of the total dose of the psychedelic compound to the patient within about 8 minutes to about 25 minutes, or from about 20% to about 55% of the total dose of the psychedelic compound to the patient within about 10 minutes to about 23 minutes, or from about 25% to about 50% of the total dose of the psychedelic compound to the patient within about 12 minutes to about 22 minutes, or from about 30% to about 45% of the total dose of the psychedelic compound to the patient within about 15 minutes to about 20 minutes. The remainder of the total dose may be administered at a different average infusion rate.

When the blood plasma concentration of psilocin in the patient is monitored, the rate of administration of the psychedelic compound may be adjusted to maintain the blood plasma concentration of psilocin in the patient at between about 5 ng/mL to about 10 ng/mL for over half of the total time of the administration.

### Compositions

The method may comprise administering a pharmaceutical composition comprising a therapeutically effective amount of the psychedelic compound to a patient by intravenous infusion. The pharmaceutical composition is typically a formulation suitable for intravenous infusion. For example, the pharmaceutical composition may comprise the psychedelic compound and one or more pharmaceutically acceptable carriers, solvents, diluents, adjuvants, excipients, or vehicles. The pharmaceutical composition may comprise the psychedelic compound and one or more pharmaceutically acceptable carriers, solvents, solubilisers, diluents, adjuvants, excipients, or vehicles. The pharmaceutical composition is typically a solution in which the psychedelic compound is dissolved, for instance an aqueous solution comprising the psychedelic compound. The psychedelic compound is typically dissolved in a diluent. The pharmaceutical composition may further comprise one or more of a buffer, a solubilizer, a pH modifier, a surfactant, an anti-oxidant, a chelating agent, or a cyclodextrin. Typically, the pharmaceutical composition is a formulation comprising the psychedelic compound, at least one pharmaceutically acceptable diluent, and optionally further comprising at least one pharmaceutically acceptable solubiliser. Preferably, the pharmaceutical composition is a solution comprising the psychedelic compound, at least one pharmaceutically acceptable diluent, and optionally further comprising at least one pharmaceutically acceptable solubiliser.

Typical parenteral media may be used, for example water for injection, saline, dextrose etc.

The diluent is typically water. The solubiliser is typically a cyclodextrin, for example 2-hydroxypropyl-β-cyclodextrin.

The formulation may comprise the psychedelic compound at a concentration of at least about 50 mg/g relative to the total weight of the formulation. Typically, the formulation comprises the psychedelic compound at a concentration of at least about 60 mg/g, at least about 70 mg/g, at least about 80 mg/g, at least about 90 mg/g, at least about 100 mg/g, at least about 125 mg/g, at least about 150 mg/g, at least about 175 mg/g, at least about 200 mg/g, or at least about 225 mg/g. Preferably, the formulation comprises the psychedelic compound at a concentration of at least about 70 mg/g, or at least about 80 mg/g, or at least about 90 mg/g. The formulation may comprise the psychedelic compound at a concentration of no more than about 400 mg/g, preferably no more than about 300 mg/g. For instance, the concentration of the psychedelic compound may be from 70 to 300 mg/g relative to the total weight of the formulation. The concentration of the psychedelic compound may be from 70 to 100 mg/g, from 100 to 130 mg/g, from 130 to 170 mg/g, from 170 to 220 mg/g or from 220 to 300 mg/g.

Alternatively, the formulation may comprise the psychedelic compound at a concentration of from 1 to 10 mg/g, for example from 1 to 8 mg/g, from 1 to 6 mg/g, from 2 to 5 mg/g, or from 3 to 4 mg/g. The concentration of the psychedelic compound may be from 2 to 4 mg/g, for example about 3 mg/g. The concentration of the psychedelic compound may be from 3 to 5 mg/g, for example about 4 mg/g.

When the psychedelic compound is psilocybin or a pharmaceutically acceptable salt thereof, typically the formulation comprises the psychedelic compound at a concentration of at least about 3 mg/g, at least about 60 mg/g, at least about 70 mg/g, at least about 80 mg/g or at least about 90 mg/g. Preferably, when the psychedelic compound is psilocybin or a pharmaceutically acceptable salt thereof, the formulation comprises psilocybin at a concentration of at least about 70 mg/g, more preferably at least about 80 mg/g. When the psychedelic compound is psilocybin or a pharmaceutically acceptable salt thereof, the formulation may comprise psilocybin at a concentration of about 90 mg/g, for instance from 80 to 100 mg/g.

When the psychedelic compound is psilocin or a pharmaceutically acceptable salt thereof, typically the formulation comprises the psychedelic compound at a concentration of at least about 4 mg/g, at least about 150 mg/g, at least about 175 mg/g, at least about 200 mg/g, at least about 225 mg/g, or at least about 250 mg/g. Preferably, when the psychedelic compound is psilocin or a pharmaceutically acceptable salt thereof, the formulation comprises psilocin at a concentration of at least about 200 mg/g, more preferably at least about 225 mg/g. When the psychedelic compound is psilocin or a pharmaceutically acceptable salt thereof, the formulation may comprise psilocin at a concentration of about 250 mg/g, for instance from 230 to 270 mg/g.

The formulation may have a pH of from pH 3 to pH 9. Typically, the formulation has a pH of from pH 4 to pH 8, for example a pH of from pH 4 to pH 6, or from pH 6 to pH 8. When the formulation is a solution and the psychedelic compound is psilocin or a pharmaceutically acceptable salt thereof, typically the pH is from pH 3 to pH 5, and is preferably about pH 4.

The formulation may be a solution comprising the psychedelic compound, water, and 2-hydroxypropyl-β-cyclodextrin, wherein the psychedelic compound is psilocin. The formulation may comprise 2-hydroxypropyl-β-cyclodextrin at a concentration of from 5 to 15% w/v, and water at a concentration of from 85 to 95% w/v. Preferably, the formulation comprises 2-hydroxypropyl-s-cyclodextrin at a concentration of from 8 to 12% w/v, and water at a concentration of from 88 to 92% w/v. More preferably, the formulation comprises 2-hydroxypropyl-β-cyclodextrin at a concentration of from 8 to 12% w/v, and water at a concentration of from 88 to 92% w/v, wherein psilocin is present at a concentration of from 230 to 270 mg/g.

The formulation may be a solution comprising the psychedelic compound and water, wherein the psychedelic compound is psilocin. The formulation may consist of water and psilocin. The formulation may comprise at least 85% w/v water, The formulation may therefore comprise 100% w/v water. Preferably, the formulation comprises 100% w/v water, wherein psilocin is present at a concentration of from 230 to 270 mg/g.

Concentrations of an excipient as used herein refer to the amount of that excipient relative to the total amount of excipient (i.e. excluding the psychedelic compound).

The formulations comprising psilocin as described herein are typically stable for 7 days at 2-8° C. As used herein, “stable” refers to stability with respect to chemical degradation of the psychedelic compound.

Typically, the method of the invention comprises administering a pharmaceutical composition comprising a therapeutically effective amount of the psychedelic compound to a patient by intravenous infusion, which pharmaceutical composition is a formulation as described herein.

When the method of the invention comprises administering a pharmaceutical composition to a patient by intravenous infusion, the method may further comprise producing the formulation suitable for intravenous infusion by (a) defrosting a frozen solution, (b) reconstituting a lyophilised product, (c) solubilising a powder for infusion, or (d) diluting a concentrate for infusion.

Suitable carriers, solvents, diluents, adjuvants, excipients, vehicles, buffers, solubilizers, pH modifiers, surfactants, anti-oxidants, chelating agents, and cyclodextrins are generally well known to the skilled person, and can be found in standard pharmaceutical texts.

### Methods of Treatment

The method of the present invention is suitable for treating any disease or condition that psychedelic compounds, such as psilocybin or psilocin, may be used to treat.

In one embodiment, the invention provides a method of treating or preventing a disease or condition selected from psychological, neurological and central nervous system disorders.

The disease or condition may be selected from: disruptive mood dysregulation disorder, depression, major depressive disorder (MDD), treatment-resistant depression, persistent depressive disorder (dysthymia), demoralization, hopelessness, premenstrual dysphoric disorder, substance/medication-induced depressive disorder, post-partum depression, depressive disorder due to another medical condition, separation anxiety disorder, selective mutism, specific phobia, social anxiety disorder (social phobia), panic disorder, panic attack, agoraphobia, generalized anxiety disorder, anxiety, death anxiety, substance-medication-induced anxiety disorder, anxiety disorder due to another medical condition, somatic symptom disorder, illness anxiety disorder (hypochondriac), conversion disorder (functional neurological symptom disorder), factitious disorder, post-traumatic stress disorder (PTSD), adjustment disorders, acute distress disorder, obsessive-compulsive disorder, body dysmorphic disorder, hoarding disorder, trichotillomania (hair-pulling) disorder, excoriation (skin-picking) disorder, substance/medication-induced obsessive-compulsive and related disorder, obsessive-compulsive and related disorder due to another medical condition, substance-related disorders, alcohol-related disorders, cannabis-related disorders, hallucinogen-related disorders, inhalant-related disorders, cocaine-related disorders, opioid-related disorders, sedative-, hypnotic-, or anxiolytic-related disorders, stimulant-related disorders, tobacco-related disorders, non-substance-related disorders (gambling or gaming disorder), migraines, cluster headaches (including chronic cluster headaches), cyclical vomiting, tension-type headache, dysphasia, pica, anorexia nervosa, bulimia nervosa, binge-eating disorder, oppositional defiant disorder, intermittent explosive disorder, conduct disorder, antisocial personality disorder, psychopathy, pyromania, kleptomania, autism spectrum disorder, antisocial personality disorder, attention-deficit/hyperactivity disorder, schizotypal (personality) disorder, delusional disorder, schizophrenia, schizoaffective disorder, insomnia disorder, hypersomnolence disorder, narcolepsy, primary central sleep apnea, bipolar I disorder, bipolar II disorder, cyclothymic disorder, pain, phantom pain, chronic pain, myelopathy, traumatic brain injury, intellectual disabilities, mania, neurodegeneration, paraphilic disorders (e.g., paedophilic disorder), suicidal behavior disorder, suicidal ideation, desire for hastened death, non-suicidal self-injury, persistent complex bereavement disorder, epilepsy, locked-in syndrome and restless leg syndrome.

In one embodiment, the method is a method of treating or preventing a disease or condition selected from depression, anxiety, death anxiety, demoralization, adjustment disorders, hopelessness, suicidal ideation and desire for hastened death.

In one embodiment, the method is a method of treating or preventing cocaine-related disorders, opioid-related disorders, or stimulant-related disorders.

The method may be a method of treating or preventing depression in a patient. The method may be a method of treating or preventing anxiety in a patient. As used herein, treating or preventing depression and/or anxiety includes reducing the symptoms of depression and/or anxiety or achieving remission of depression and/or anxiety. In one embodiment, treating or preventing depression and/or anxiety comprises reducing the symptoms of depression and/or anxiety. The patient may report a reduction of symptoms of depression and/or anxiety.

In one embodiment, the patient has been identified as being in need of treatment to alleviate depression and/or anxiety. In one embodiment, the patient has indicated that he or she is suffering from depression and/or anxiety.

The symptoms of depression and/or anxiety may be measured using the Hospital Anxiety and Depression Scale (HADS; Zigmond and Snaith (1983), “The hospital anxiety and depression Scale”, Acta Psychiatrica Scand, 67: 361-370). In this test, lower numbers indicate lower levels of depression and/or anxiety. Subscale scores can be calculated for depression (HADS-D) and anxiety (HADS-A). A subscale score equal to or above 8 and a full scale score over 12 indicates the possible presence of a clinical disorder.

Accordingly, in the method of the invention a total Hospital Anxiety and Depression Scale score of the patient may be reduced after administration of the therapeutically effective amount of the psychedelic compound. In one embodiment, the total HADS score of the patient is reduced to below about 12 after administration of the therapeutically effective amount of the psychedelic compound.

The severity of depression may also be measured using the Beck Depression Inventory-II (BDI-II; Beck et al (1988), “Psychometric properties of the Beck Depression Inventory: Twenty-five years of evaluation”, Clin Psych Rev, 8: 77-100). Scores above 12 indicate possible clinical depression.

Accordingly, in the method of the invention a Beck Depression Inventory-II score of the patient may be reduced after administration of the therapeutically effective amount of the psychedelic compound. In one embodiment, the Beck Depression Inventory-II score of the patient is reduced to below about 12 after administration of the therapeutically effective amount of the psychedelic compound.

The method may be a method of treating or preventing death anxiety. The method may be a method of treating or preventing demoralization (i.e. loss of meaning in life). The method may be a method of treating or preventing hopelessness. Death anxiety, demoralization and hopelessness are aspects of existential distress. Thus, the method may also be a method of treating or preventing existential distress in a patient, wherein treating or preventing existential distress includes reducing levels of at least one of death anxiety, hopelessness and demoralization.

In the method of treating or preventing death anxiety in a patient, death anxiety is reduced relative to the death anxiety of the patient before the administration of the therapeutically effective amount of the psychedelic compound. Death anxiety is typically measured according to the Death Anxiety Scale (Templer (1970), “The construction and validation of a death anxiety scale”, J Gen Psychol, 82: 165-177). Scores below 8 are considered normative levels of death anxiety. Accordingly, in the method of the invention a death anxiety score of the patient may be reduced to less than 8 after administration of the therapeutically effective amount of the psychedelic compound.

In the method of treating or preventing demoralization in a patient, demoralization is reduced relative to the demoralization of the patient before the administration of the therapeutically effective amount of the psychedelic compound. Demoralization is typically measured according to the Demoralization Scale (Kissane et al. (2004), “The demoralization scale: A report of its development and preliminary validation”, J Palliat Care, 20: 269-276). Scores above 30 are considered indicative of clinical levels of demoralization. Accordingly, in the method of the invention a demoralization score of the patient may be reduced to less than 30 after administration of the therapeutically effective amount of the psychedelic compound.

In the method of treating or preventing hopelessness in a patient, hopelessness is reduced relative to the hopelessness of the patient before the administration of the therapeutically effective amount of the psychedelic compound. Hopelessness is typically measured according to the Hopelessness Assessment in Illness instrument (Rosenfeld et al. (2011), “Assessing hopelessness in terminally ill cancer patients: Development of the Hopelessness Assessment in Illness Questionnaire”, Psychol Assess, 23: 325-336), on a scale of 0-16. Higher scores indicate higher levels of hopelessness. Accordingly, in the method of the invention a Hopelessness Assessment in Illness score of the patient may be reduced to less than 8 after administration of the therapeutically effective amount of the psychedelic compound.

The method may be a method of treating or preventing suicidal ideation in a patient. As used herein, treating or preventing suicidal ideation includes reducing or preventing suicidal thinking, suicidal planning and/or suicide attempts. The patient may report a reduction in suicidal thinking and/or suicidal planning. The patient may make less frequent suicide attempts.

In one embodiment, the patient has been identified as being in need of treatment to prevent or reduce suicidal ideation. Accordingly, the method of the invention may include a step of assessing the level of suicidal ideation in the patient prior to administering the therapeutically effective amount of the psychedelic compound to said patient. In one embodiment, the patient has indicated that he or she is suffering from suicidal ideation.

Suicidal ideation may be measured using a composite test comprising elements from the Beck Depression Inventory-II (BDI-II; Beck et al (1988), “Psychometric properties of the Beck Depression Inventory: Twenty-five years of evaluation”, Clin Psych Rev, 8: 77-100) and the Brief Symptom Inventory (BSI; Derogatis 1993). In the BDI, Item #9 queries suicidal ideation with the following options: 0=I don't have any thoughts of killing myself; 1=I have thoughts of killing myself, but I would not carry them out; 2=I would like to kill myself; 3=I would kill myself if I had the chance. In the BSI, item #9 (“Thoughts of ending your life”) also correlates to suicidal ideation, and is measured on a Likert scale: 0=Not at all; 1=Little; 2=Moderately; 3=Quite a bit; 4=Extremely. The aggregate composite suicidal ideation score is calculated by adding the scores from BDI-II item #9 to BSI Item #9. The composite score may be calculated by computing Z-scores for each item and summing them, and then the composite Z-scores may be transformed into standardized T-scores with a range of 0 to 100 (Song et al., 2013). Higher scores indicate higher SI.

Accordingly, in the method of the invention a composite suicidal ideation score of the patient may be reduced after administration of the therapeutically effective amount of the psychedelic compound. Typically, a composite suicidal ideation score of the patient is reduced by at least 20%, at least 30%, at least 40%, at least 50% or at least 75% after administration of the therapeutically acceptable amount of the psychedelic compound. In one embodiment, a composite suicidal ideation score of the patient after administration of the therapeutically effective amount of the psychedelic compound is less than 50, less than 45 or less than 40.

The method may be a method of treating or preventing desire for hastened death in a patient. As used herein, treating or preventing desire for hastened death includes preventing or reducing the desire for a more rapid death than would naturally occur. The patient may report a reduction in desire for a more rapid death than would naturally occur.

In one embodiment, the patient has been identified as being in need of treatment to prevent or reduce desire for hastened death. Accordingly, the method of the invention may include a step of assessing the level of desire for hastened death in the patient prior to administering the therapeutically effective amount of the psychedelic compound to said patient. In one embodiment, the patient has indicated that he or she is suffering from desire for hastened death.

Desire for hastened death may be measured using the schedule of attitudes towards hastened death (SAHD) (Rosenfeld 2000). The SAHD is a 20-item true/false measure of desire for hastened death, which has been validated in patients with cancer. Alternatively, DHD can be measured using the loss of meaning factor from the Demoralization Scale (Kissane et al. (2004)). In particular, a composite desire for hastened death score can be created from the following five items from the loss of meaning factor, as measured on a Likert scale from zero to four: “Life is no longer worth living”, “I would rather not be alive”, “My life seems to be pointless”, “My role in life has been lost”, and “There is no purpose to the activities in my life”.

Accordingly, in the method of the invention a composite desire for hastened death score of the patient may be reduced after administration of the therapeutically effective amount of the psychedelic compound. Typically, a composite desire for hastened death score of the patient is reduced by at least 20%, at least 40%, at least 60% or at least 80% after administration of the therapeutically effective amount of the psychedelic compound.

The patient to be treated may be suffering from a life-threatening disease. The life-threatening disease may be any chronic disease which has the potential to reduce the normal life expectancy of a patient suffering from the disease. The life-threatening disease may be selected from cancer, heart disease, chronic obstructive pulmonary disease (COPD), diabetes mellitus, Alzheimer's, dementia, motor neurone disease, amyotrophic lateral sclerosis (ALS), Parkinson's disease, epilepsy, multiple sclerosis, and myalgic encephalopathy (ME). In one embodiment, the life-threatening disease is cancer.

Also provided by the invention is a psychedelic compound for use in a method of treating or preventing a disease or condition as described herein in a patient, wherein the psychedelic compound is psilocybin or psilocin, or a pharmaceutically acceptable salt thereof; and the method comprises administering a therapeutically effective amount of a psychedelic compound to the patient by intravenous infusion.

In one embodiment, the invention provides a psychedelic compound as described herein, for use in the treatment of a disease or condition selected from depression, anxiety, death anxiety, demoralization, adjustment disorders, hopelessness, suicidal ideation and desire for hastened death. In one embodiment, the disease or condition is selected from cocaine-related disorders, opioid-related disorders and stimulant-related disorders.

The invention further provides use of a psychedelic compound in the manufacture of a medicament for use in a method of treating or preventing of a disease or condition in a patient, wherein the psychedelic compound is psilocybin or psilocin, or a pharmaceutically acceptable salt thereof; and the method comprises administering a therapeutically effective amount of a psychedelic compound to the patient by intravenous infusion. The disease or condition may be selected, for example, from depression, anxiety, death anxiety, demoralization, adjustment disorders, hopelessness, suicidal ideation and desire for hastened death. In one embodiment, the disease or condition is selected from cocaine-related disorders, opioid-related disorders and stimulant-related disorders. The treatment or prevention of the disease or condition may be as described herein.

### Kits

The invention also provides a kit comprising one or more infusion bags, syringes, vials and/or ampoules comprising a formulation suitable for administration by intravenous infusion, which formulation comprises a psychedelic compound which is psilocybin or psilocin, or a pharmaceutically acceptable salt thereof; and instructions for use of the one or more infusion bags, syringes, vials and/or ampoules in a method as defined herein.

In one embodiment, the kit comprises one or more syringes which are intended to be used with a syringe driver during administration of the psychedelic compound to a patient.

In another embodiment, the kit comprises one or more infusion bags through the psychedelic compound may be administered to the patient under gravity. In this embodiment, the kit may further comprise one or more clamps, which may be utilised to assist in controlling the rate of administration of the psychedelic compound to the patient.

In another embodiment, the kit comprises one or more vials comprising a formulation comprising a psychedelic compound which is psilocybin or psilocin, or a pharmaceutically acceptable salt thereof.

In another embodiment, the kit comprises one or more ampoules comprising a formulation comprising a psychedelic compound which is psilocybin or psilocin, or a pharmaceutically acceptable salt thereof.

### Devices

The invention also provides a device which is configured to administer a psychedelic compound to a patient by intravenous infusion, wherein the psychedelic compound is psilocybin or psilocin, or a pharmaceutically acceptable salt thereof; and administering the psychedelic compound to a patient by intravenous infusion is as defined herein. The device may, for example, be a syringe driver, a syringe, or an infusion bag. The device may be a smart infusion pump, which is configured to monitor the blood plasma concentration of psilocin and administer the psychedelic compound only when the concentration of psilocin drops below a predetermined value. The predetermined value may be, for example, about 5 ng/mL.

The invention is described in more detail by the following Examples. Although preferred embodiments have been depicted and described in detail herein, it will be apparent to those skilled in the relevant art that various modifications, additions, substitutions and the like can be made without departing from the spirit of the invention and these are therefore considered to be within the scope of the invention as defined in the claims which follow.

The texts of references cited in this disclosure are herein incorporated by reference in their entireties.

### EXAMPLES

### Example 1

All analysis was performed using the software R and RStudio, with models being defined using analytical and ODE solutions.

Systemic PK parameters were derived for published human bolus psilocybin IV data and human infusion simulations were performed over a range of doses to identify infusion regimens that achieved the target profile.

Human IV PK data for psilocin after psilocybin was administered was taken from Hasler et al. “Determination of psilocin and 4-hydroxyindole-3-acetic acid in plasma by HPLC-ECD and pharmacokinetic profiles of oral and intravenous psilocybin in man”. Pharm Acta Helv. 1997 June; 72(3):175-84. Model selection was performed by assessing visual fit to data and the respective model AIC's of a one compartment and two compartment IV PK model. These systemic human PK parameters were then used for human infusion simulations by simulating the PK profiles over a range of doses. The effect of two infusion rates, a faster followed by slower infusion rate was also investigated.

### IV Modelling of Human Bolus Data

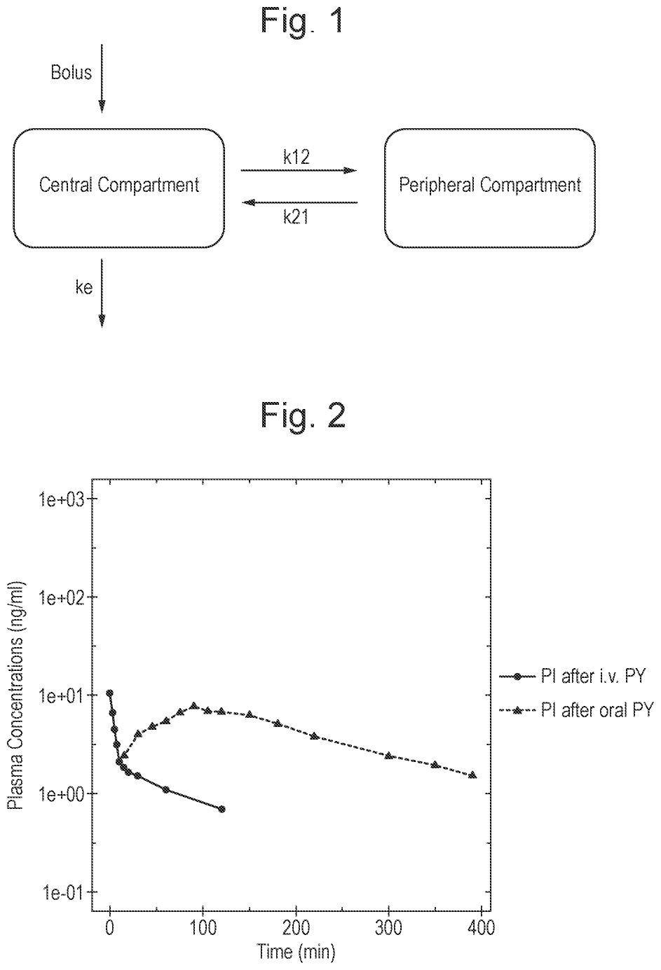

A two-compartment IV analytical model best described the data as seen in FIG. 1 and equations (1)-(6), Cl is the clearance (units mL/min), V is the volume of distribution of the central compartment (units mL), with k12, k21 (units 1/min) representing the distribution rates to and from the peripheral compartment.

$$k e = Cl / V , \qquad ( 1 )$$

$$beta = 0.5 * ( k 12 + k 21 + k e - s q r t ( {( k 12 + k 21 + k e )}^{2} - 4 * k 21 * k e ) ) , \qquad ( 2 )$$

$$alpha = k 21 * \frac{ke}{beta} , \qquad ( 3 )$$

$$A = \frac{alpha - k21}{V * ( a l p h a - b e t a )} , \qquad ( 4 )$$

$$B = \frac{beta - k 21}{V * ( be ta - alpha )} , \qquad ( 5 )$$

$$Conc = dose * ( A * exp ( - alpha * t 1 ) + B * exp ( - beta * t 1 ) ) \qquad ( 6 )$$

FIG. 2 shows visualized plasma concentration data, Hasler et al. “Determination of psilocin and 4-hydroxyindole-3-acetic acid in plasma by HPLC-ECD and pharmacokinetic profiles of oral and intravenous psilocybin in man”. Pharm Acta Helv. 1997 June; 72(3):175-84. It is assumed that the generation of psilocin is instantaneous and complete once psilocybin is absorbed. FIG. 2 shows measured exposure of psilocin after a 1 mg psilocybin IV administration and psilocin levels after a 15 mg oral dose of psilocybin.

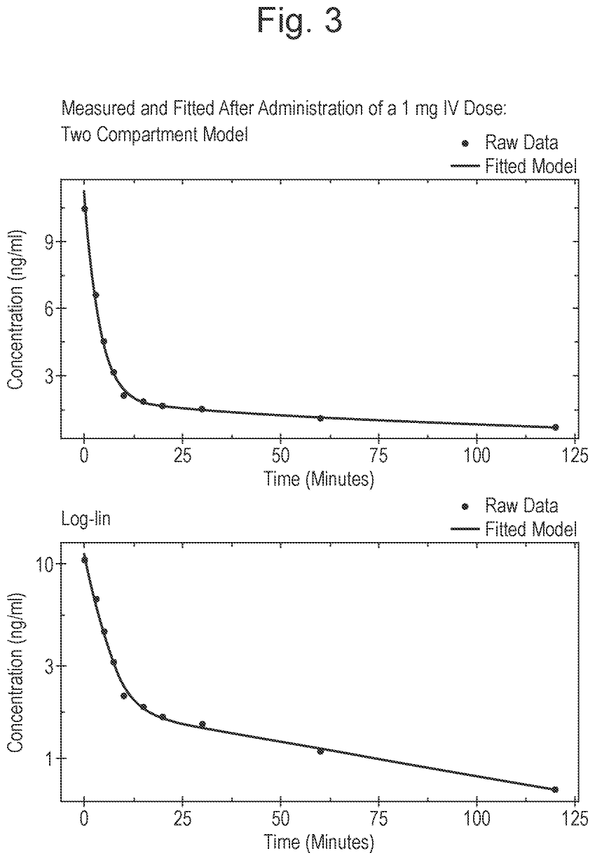

An IV two compartment model best described the IV data from FIG. 2. A two-compartment model gave a good description of the raw data capturing the Cₘₐₓ and terminal phase well, FIG. 3. The systemic PK parameters can be seen in Table 1 and there are low errors in the parameter estimates.

TABLE 1
Fitted values of two compartment IV model

| Parameter | Parameter estimate (95% CI) | |
|---|---|---|
| V (ml) | 88600 | (77300, 99900) |
| Cl (ml/min) | 3810 | (3340, 4290) |
| k12 (/min) | 0.176 | (0.144, 0.208) |
| k21 (/min) | 0.0489 | (0.0388, 0.0591) |

### Simulation Design and Results

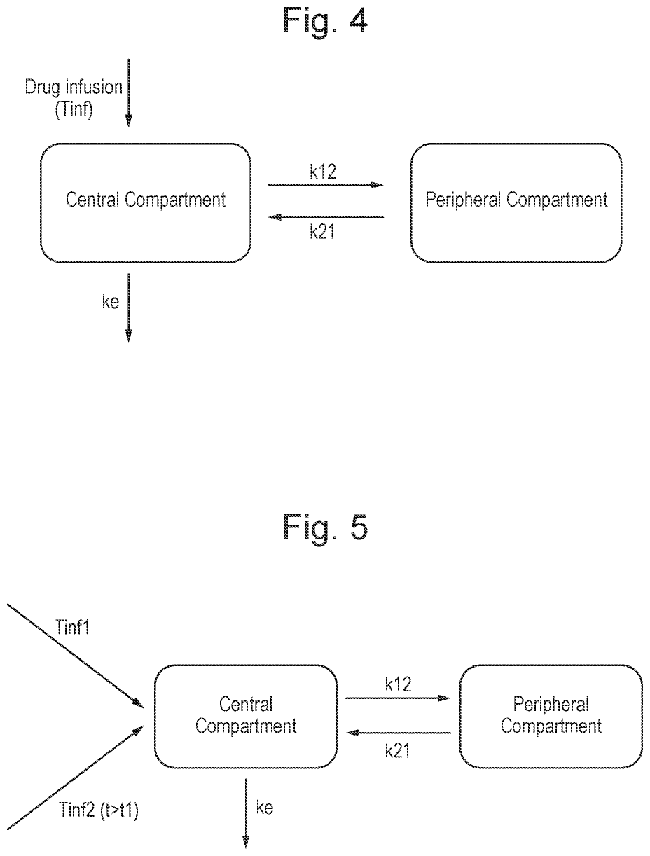

Using the human systemic PK parameters established by modelling the IV data, human IV infusion simulations were then performed using zero order infusion rates. The model schematic for IV infusion can be seen in FIG. 4 where ka is dose/Tinf and is described by equations (9)-(14). If the target profile could not be achieved with a single infusion rate and duration then multiple infusion rates and durations were investigated, the model schematic seen in FIG. 5 and equations (15)-(18) with the ka rates (zero order: mg/min) being the value dose 1 and dose 2 divided by Tinf1 and Tinf2 respectively.

$$k e = \frac{Cl}{V} , \qquad ( 9 )$$

$$beta = 0.5 * ( k 12 + k 21 + k e - s q r t ( {( k 12 + k 21 + k e )}^{2} - 4 * k 21 * k e ) ) , \qquad ( 10 )$$

$$alpha = \frac{k21 * ke}{beta} , \qquad ( 11 )$$

$$A = ( \frac{1}{V} ) * ( \frac{alpha - k 21}{alpha - beta} ) , \qquad ( 12 )$$

$$B = ( \frac{1}{V} ) * ( \frac{beta - k 21}{beta - alpha} ) , \qquad ( 13 )$$

$$Conc = ( \frac{dose}{T inf} ) * ( ( \frac{A}{a l p h a} ) * ( 1 - exp ( - alpha * t ) ) + ( \frac{B}{beta} ) * ( 1 - exp ( - beta * t ) ) ) ( if t \leq tinf ) else : \qquad ( 14 )$$

$$Conc = ( \frac{dose}{Tinf} ) * ( \begin{matrix}( \frac{A}{( alpha )} * ( 1 - exp ( - alpha * Tinf ) ) * \\ ( exp ( - alpha * ( t - Tinf ) ) ) + ( \frac{B}{beta} ) * \\ ( 1 - exp ( - beta * Tinf ) ) * ( exp ( - beta * ( t - Tinf ) ) )\end{matrix} )$$

$$\frac{d}{d t} depot 1 = - ka 1 * depot 1 , \qquad ( 15 )$$

$$\frac{d}{d t} depot 2 = - ka 2 * ( depot 2 ) * ( t > t 1 ) , \qquad ( 16 )$$

$$\frac{d}{d t} center = ( k a 1 * ( depot 1 ) + ka 2 * ( depot 2 ) * ( t > t 1 ) ) - ke * center - k 12 * center + k 21 * peri , \qquad ( 17 )$$

$$\frac{d}{d t} peri = k 12 * center - k 21 * peri , \qquad ( 18 )$$

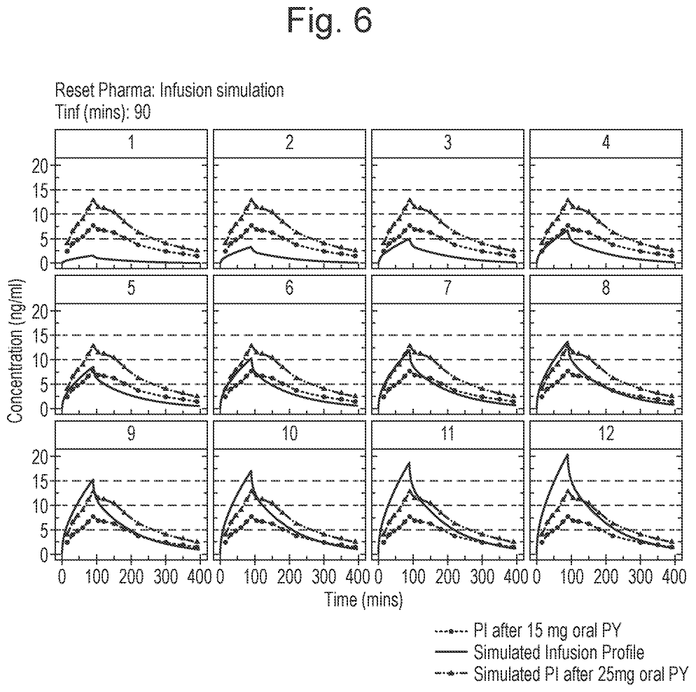

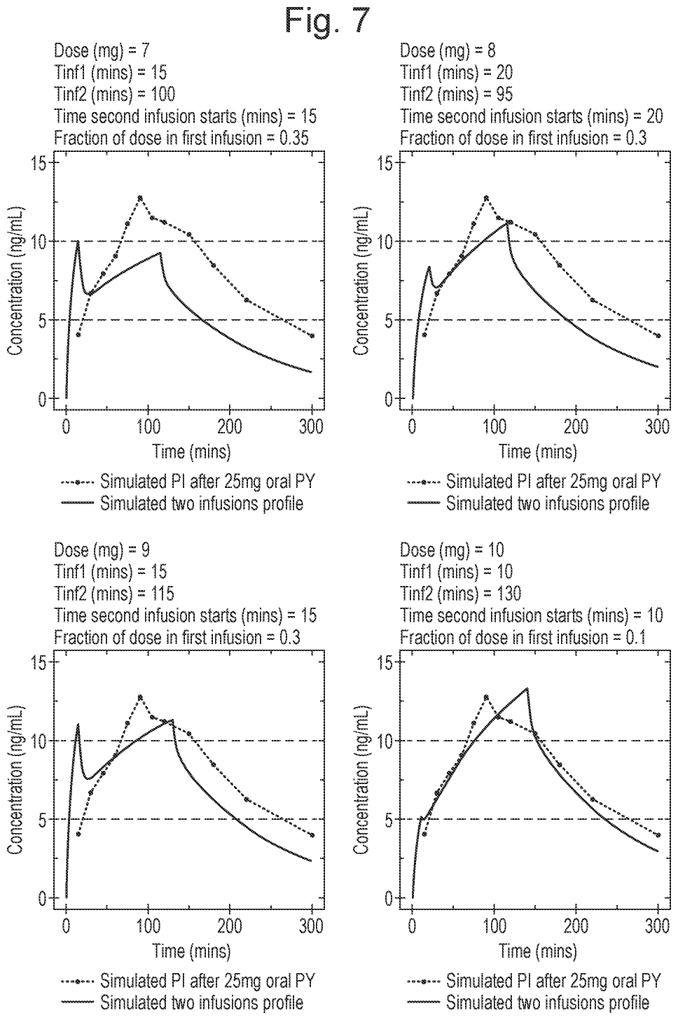

Human IV 90-minute infusion simulations were run from doses 1-12 mg (FIG. 6). The simulated profiles (blue line) reached Tₘₐₓ at 90 minutes and then are quickly cleared from the body. Table 2 shows that 8-10 mg doses give a satisfactory time to get to 5 ng/ml ranging from 12.3 to 7.7 minutes. They also give a satisfactory time of exposure greater than 5 ng/ml ranging from 167.9-200 minutes. The Cₘₐₓ's however, are all greater than 10 ng/ml with a 10 mg dose being greater than 15 ng/ml. Therefore, two infusion rates were investigated, an initial fast infusion rate (Tinf1=length of first infusion rate (min)) used to achieve therapeutic concentration (5 ng/ml) within the target time, then a second infusion rate follows immediately, in an attempt to maintain concentration above 5 and below 10 ng/ml. A number of illustrative infusion rate situations have been simulated and are shown in FIG. 7. In the simulations the second infusion rate uses the remaining dose available and runs for length Tinf2. The multiple infusion rate PK profiles can be seen in FIG. 7. It can be seen in Table 3 that using >1 infusion rate (e.g. 2 infusion rates) can give the desired PK profiles.

TABLE 2
Time to 5 ng/ml and time above 5 ng/ml after
a 90 minute IV infusion over a range of doses

| | Time to get to | Time above | Exceeds |
|---|---|---|---|
| Dose | 5 ng/ml (min) | 5 ng/ml (min) | 10 ng/ml |
| 3 | 86.3 | 4 | N |
| 4 | 50.7 | 48.1 | N |
| 5 | 33.5 | 88.8 | N |
| 6 | 23.2 | 121.6 | N |
| 7 | 16.6 | 147.2 | Y |
| 8 | 12.3 | 167.9 | Y |
| 9 | 9.5 | 185.2 | Y |
| 10 | 7.7 | 200 | Y |
| 11 | 6.4 | 213.1 | Y |
| 12 | 5.5 | 224.7 | Y |

TABLE 3
Summary table after administration with two infusion rates over a range of doses

| | | | | | | Time to | Time to | |
|---|---|---|---|---|---|---|---|---|
| | | 2^{nd} | | | | get to | drop | |
| | | infusion | | Fraction | Time to | 10 | below 5 | Exceeds |
| Dose | Tinf1 | start | Tinf2 | of dose | 5 ng/ml | ng/ml | ng/ml | 10 |
| (mg) | (min) | time | (min) | in Inf1 | (mins) | (mins) | (mins) | ng/ml |
| 7 | 15 | 15 | 100 | 0.35 | 3.977 | 14.8312 | 166.396 | N |
| 8 | 20 | 20 | 95 | 0.3 | 6.615 | 85.105 | 187.858 | Y |
| 9 | 15 | 15 | 115 | 0.3 | 3.451 | 11.9144 | 206.9688 | Y |
| 10 | 10 | 10 | 130 | 0.1 | 9.475 | 75.535 | 234.932 | Y |

### Example 2

The following Example describes the preparation of formulations suitable for administration by intravenous infusion.

### Materials

The materials used in the preparation of the formulations are as follows.

Material

Psilocybin
Psilocin
Methanol
Ethanol
Dimethyl sulfoxide (DMSO)
Dichloromethane (DCM)
N-methylpyrrolidone (NMP)
2H,3H-Decafluoropentane 98%
Orthophosphoric acid
Trifluoroacetic acid
Propylene glycol (PG)
Super refined Polysorbate 80-LQ-(MH)
(Tween 80, polyoxyethylene (80) sorbitan monooleate)
Super refined corn oil-LQ-(MH)
Super refined polyethylene glycol (PEG) 400-LQ-(MH)
Kollidon@ 17 PF (polyvinylpyrrolidone, PVP k17)
Transcutol HP (2-(2-Ethoxyethoxy)ethanol)
Labrafac lipophile WL 1349 (mixture of triglycerides
of caprylic (C₈) and capric (C₁₀) acid)
Glycerine
Labrafil M 1944 CS (oleoyl macrogol-6 glycerides)
Kolliphor ® EL (polyoxyl-35-castor oil)
Labrafac PG (Propylene glycol dicaprylocaprate)
Kolliphor ® ELP (polyoxyl-35-castor oil)
Super refined peanut oil-LQ-(MH)
Kolliphor ® HS 15 (Polyethylene glycol 660 12-hydoxystearate)
Super refined safflower USP-LQ-(MH)
Capryol 90 (Propylene glycol monocaprylate)
Sulfobutylether-β-Cyclodextrin (SBEβCD)
Cavitron ™W7 HP5 Pharma (2-Hydroxypropyl-β-cyclodextrin, HPβCD)
Vitamin E TPGS
PEG 1500
Poloxamer 188 (Polyethylene-polypropylene glycol)
Hydrochloric acid 5M
Glacial acetic acid

### Preparative Work

The solubility of non-micronised psilocybin and psilocin was first assessed in the following 20 excipients:

| DMSO | NMP |
|---|---|
| Ethanol | Propylene glycol |
| Tween 80 | PEG 400 |
| Transcutol HP | Glycerine |
| Kolliphor EL | Kolliphor HS 15 (10% aq. solution) |
| Capryol 90 | Labrafac lipophile WL 1349 |
| Labrafil M 1944 CS | Labrafac PG |
| Peanut oil | Safflower oil |
| Poloxamer 188 (10% aq. solution) | SBEβCD (10% aq. solution) |
| HPβCD (10% aq. solution) | Acetic acid (1% aq. solution) |
| PEG 1500 (10% aq. solution) | Vitamin E TPGS (10% aq. solution) |

Due to the poor solubility of psilocybin observed in all the excipients tested, solution development was not taken further.

Psilocin was shown to have good solubility in DMSO and NMP. Solution development was investigated with psilocin at a concentration of 30 mg/g in different aqueous vehicles (PEG/Transcutol HP/HPβCD, DMSO/PEG 400/Kolliphor ELP) and one non-aqueous vehicle (DMSO/PEG 400/Kolliphor ELP, peanut oil). Initial trials illustrated that psilocin had higher solubility at acidic pH, and so based on the initial results the feasibility to develop solutions at higher concentrations of 200-250 mg/g psilocin, using low pH (pH 4), was assessed in the aqueous vehicles. Lead formulations were selected based on solubility and stability measurements.

### Formulations Comprising Psilocin

Vehicle components were prepared as weight per volume in a volumetric flask. The solutions were then prepared at a 1 g scale by weighing 250 mg of psilocin in a 4 mL glass vial and 400 mg of vehicle. The mixtures were stirred whilst adding small aliquots of 5 M HCl by weight until pH 4 was achieved. Depending on the amount of HCl added to achieve pH 4, further vehicle was added to achieve a concentration of 250 mg/g psilocin.

The formulations prepared are described in Table 4.

TABLE 4
Formulations comprising psilocin

| | Composition |
|---|---|
| Formulation 1 | 250 mg/g psilocin in 10% HPβCD (aq.) |
| Formulation 2 | 250 mg/g psilocin in 100% water |

### Example 3

The following Example demonstrates the chemical and physical stability of the formulations of Example 2.

### Analytical Methods

### Equipment

The following equipment was used in Example 3.

| Instrument/Equipment | Series | Manufacturer |
|---|---|---|
| HPLC-UV | 1100 | Agilent |
| pH-meter | SevenCompact | Mettler Toledo |
| pH electrode | In-Lab Micro | Mettler-Toledo |
| Heater/shaker | RS9000 | Barnstead STEM ™ |
| Osmometer | Osmomat ™ 3000 | Gonotec ® |

### Psilocin Analysis by HPLC-UV

The HPLC-UV method used for the analysis of psilocin is detailed in Table 5. For each HPLC experiment, two independent standards (A and B) were prepared at a psilocin (API) concentration of 0.1 mg/mL in 10 mM HCl (diluent). Approximately 2 mg of psilocin was weighed into a 20 mL volumetric flask, followed by the addition of the diluent up to 20 mL and then sonicated until complete dissolution of API (approximately 10 minutes). The solutions were transferred to HPLC vials in preparation for HPLC analysis.

TABLE 5
Summary of the HPLC method used for psilocin

| Column | Ascentis ® Express C18 | | |
|---|---|---|---|
| | (100 × 4.6 mm, 2.7 μm) | | |
| | Cat#53827-U | | |
| | Col: USPY010599 | | |
| Flow rate (mL/min) | 1 | | |
| Injection volume (μL) | 6 | | |
| Autosampler tray temperature (° C.) | 25 | | |
| Diluent | 10 mM HCl | | |
| Detection wavelength (nm) | 220 | | |
| Run time (min) | 25 | | |
| Standard concentration (mg/mL) | 0.1 | | |
| Mobile phase A | 0.05% TFA in water | | |
| Mobile phase B | Acetonitrile | | |
| Column temperature (° C.) | 30 | | |
| | Gradient | | |
| | Time | Mobile | Mobile |
| | (min) | phase A % | phase B % |
| Elution mode | 1 | 94 | 6 |
| | 10 | 82 | 18 |
| | 15 | 25 | 75 |
| | 20 | 25 | 75 |
| | 20.1 | 94 | 6 |
| | 25 | 94 | 6 |

### Stability Testing of Formulations Comprising Psilocin

Formulations 1 and 2 were stored in closed glass vials at 2-8° C. for stability testing. At the following time-points: t=0, 1, 5 and 7 days, the chemical and physical stability of the solutions was assessed visually, by measuring the pH and osmolality, and by assay.

To assay the solutions, 50 μL of the solution was first centrifuged at 12,500 rpm for 10 minutes. Next, 10 μL of the supernatant was weighed into a 25 mL volumetric flask, in duplicate, and the volume was filled with 10 mM HCl. The volumetric flasks were then vortexed for approximately 30 seconds and then transferred to HPLC vials for assay and purity quantification.

### Results

The results are shown in Table 6.

TABLE 6
Stability results for formulations 1 and 2

| | Time- | | | | Measured | |
|---|---|---|---|---|---|---|
| | point/storage | | Osmolality | Assay | concentration | Purity |
| Formulation | condition | pH | (mOsm/kg) | (%) | (mg/g) | (%) |
| 1 | t = 0 | 4.2 | 1521 | 110.9 ± | 278.2 ± 5.9 | 99.2 |
| | | | | 2.3 | | |
| | t = 24 | 3.9 | 1525 | 116.4 ± | 292.0 ± 1.2 | 99.2 |
| | h/2-8° C. | | | 0.5 | | |
| | t = 5 | 3.7 | 1526 | 125.4 ± | 314.5 ± 31.4 | 99.1 |
| | d/2-8° C. | | | 12.5 | | |
| | t = 7 | 3.7 | 1532 | 112.5 ± | 282.2 ± 19.4 | 99.2 |
| | d/2-8° C. | | | 7.7 | | |
| 2 | t = 0 | 4.2 | 1211 | 114.4 ± | 287.3 ± 8.6 | 99.2 |
| | | | | 3.4 | | |
| | t = 24 | 4.3 | 1212 | 103.3 ± | 259.3 ± 11.9 | 99.2 |
| | h/2-8° C. | | | 4.7 | | |
| | t = 5 | 4.1 | 1205 | 108.9 ± | 273.5 ± 5.5 | 99.2 |
| | d/2-8° C. | | | 2.2 | | |
| | t = 7 | 4.2 | 1207 | 105.9 | 265.9 | 99.2 |
| | d/2-8° C. | | | | | |

In conclusion, the solutions were chemically and physically stable upon storage at 2-8° C. for at least 7 days. There was no reduction over time in the percentage peak area as measured by HPLC (i.e. no degradation). It is thought that adjusting the pH of the formulations to pH 4 prevents degradation of psilocin.

### Conclusion

It has been shown that the formulations described herein are advantageously suitable for administration by intravenous infusion, and show good chemical and physical stability.

### Example 4

The dissolved fraction of psilocybin and psilocin in suspensions with different vehicles was investigated. The results are shown in Table 7.

TABLE 7
Dissolved fraction assays of psilocybin
and psilocin suspensions up to 7 days

| | Time-point/storage | Dissolved |
|---|---|---|
| Sample | condition | fraction (mg/g) |
| 90 mg/g psilocybin in | t = 0 | 8.4 ± 0.09 |
| 0.5% PVP k17, 0.1% | t = 24 h/25° C. | 8.3 ± 0.04 |
| Tween 80 in PBS | t = 4 d/25° C. | 8.6 ± 0.04 |
| | t = 7 d/25° C. | 9.3 ± 0.07 |
| 90 mg/g psilocybin | t = 0 | 9.2 ± 0.1 |
| in 10% HPβCD in PBS | t = 24 h/25° C. | 9.3 ± 0.3 |
| | t = 4 d/25° C. | 9.5 ± 0.3 |
| | t = 7 d/25° C. | 10.6 ± 0.4 |
| 90 mg/g psilocybin | t = 0 | 8.5 ± 0.08 |
| in 2% Poloxamer | t = 24 h/25° C. | 8.6 ± 0.04 |
| 188 in PBS | t = 4 d/25° C. | 8.6 ± 0.03 |
| | t = 7 d/25° C. | 9.5 ± 0.1 |
| 100 mg/g non-micronized | t = 0 | 7.4 |
| psilocybin in 10% | | |
| HPβCD in water | | |
| 250 mg/g psilocin in | t = 24 h/2-8° C. | 6.4 ± 0.1 |
| 80% corn oil, 10% NMP, | t = 5 d/2-8° C. | 7.0 ± 0.1 |
| 10% Kolliphor ELP | t = 7 d/2-8° C. | 6.6 ± 0.0003 |
| 250 mg/g psilocin in | t = 0 | 4.3 |
| 10% HPβCD in water | | |
| 250 mg/g psilocin in | t = 0 | 2.8 |
| 0.5% PVP k17 + 0.1% | | |
| Tween 80 in water | | |
| 250 mg/g psilocin in 2% | t = 0 | 3.3 |
| Poloxamer 188 in water | | |

It is expected that the suspensions comprising psilocybin would be solutions at concentrations of psilocybin of about 4 mg/g or less; and that the suspensions comprising psilocin would be solutions at concentrations of psilocybin of about 3 mg/g or less.

1. A method of treating or preventing a disease or condition in a patient, the method comprising administering a therapeutically effective amount of a psychedelic compound to the patient by intravenous infusion,
wherein the psychedelic compound is psilocybin or psilocin, or a pharmaceutically acceptable salt thereof.

2. A method according to claim 1, wherein the psychedelic compound is psilocybin or a pharmaceutically acceptable salt thereof.

3. A method according to claim 1, wherein the psychedelic compound is psilocin or a pharmaceutically acceptable salt thereof.

4. The method according to any one of the preceding claims, wherein administering the therapeutically effective amount of a psychedelic compound to a patient by intravenous infusion comprises intravenously infusing the patient for a total time of from about 5 minutes to about 200 minutes, from about 45 minutes to about 180 minutes, or from about 60 minutes to 150 minutes.

5. The method according to any one of the preceding claims, wherein administering the therapeutically effective amount of a psychedelic compound to a patient by intravenous infusion comprises intravenously infusing the patient for a total time of from about 70 minutes to about 80 minutes, about 80 minutes to about 90 minutes, about 90 minutes to about 100 minutes, about 100 minutes to about 110 minutes, about 110 minutes to about 120 minutes, about 120 minutes to about 130 minutes, about 130 minutes to about 140 minutes, about 140 minutes to about 150 minutes, or about 150 minutes to about 160 minutes.

6. The method according to any one of the preceding claims, wherein the therapeutically effective amount of the psychedelic compound is from about 1 mg to about 15 mg, from about 5 mg to about 13 mg, or from about 8 mg to about 11 mg.

7. The method according to any one of the preceding claims, wherein the therapeutically effective amount of the psychedelic compound is from about 5.5 mg to about 6.5 mg, from about 6.5 mg to about 7.5 mg, from about 7.5 mg to about 8.5 mg, from about 8.5 mg to about 9.5 mg, from about 9.5 mg to about 10.5 mg, from about 10.5 mg to about 11.5 mg, or from about 11.5 mg to about 12.5 mg.

8. The method according to any one of the preceding claims, wherein administering the therapeutically effective amount of the psychedelic compound to the patient by intravenous infusion achieves a maximum blood plasma concentration (Cₘₐₓ) of psilocin in the patient which is at least about 5 ng/mL, at least about 6 ng/mL, at least about 7 ng/mL, at least about 8 ng/mL, at least about 9 ng/mL, or at least about 10 ng/mL.

9. The method according to any one of the preceding claims, wherein administering the therapeutically effective amount of the psychedelic compound to the patient by intravenous infusion achieves a maximum blood plasma concentration (Cₘₐₓ) of psilocin in the patient which is no greater than about 20 ng/mL, no greater than about 19 ng/mL, no greater than about 18 ng/mL, no greater than about 17 ng/mL, no greater than about 16 ng/mL, or no greater than about 15 ng/mL.

10. The method according to any one of the preceding claims, wherein administering the therapeutically effective amount of the psychedelic compound to the patient by intravenous infusion achieves a maximum blood plasma concentration (Cₘₐₓ) of psilocin in the patient which is from about 6 ng/mL to about 7 ng/mL, from about 7 ng/mL to about 8 ng/mL, from about 8 ng/mL to about 9 ng/mL, from about 9 ng/mL to about 10 ng/mL, from about 10 ng/mL to about 11 ng/mL, from about 11 ng/mL to about 12 ng/mL, from about 12 ng/mL to about 13 ng/mL, from about 13 ng/mL to about 14 ng/mL, from about 14 ng/mL to about 15 ng/mL, from about 15 ng/mL to about 16 ng/mL, from about 16 ng/mL to about 17 ng/mL, from about 17 ng/mL to about 18 ng/mL, from about 18 ng/mL to about 19 ng/mL, or from about 19 ng/mL to about 20 ng/mL.

11. The method according to any one of the preceding claims, wherein the rate of infusion of the psychedelic compound varies over time.

12. The method according to any one of the preceding claims, wherein administering the therapeutically effective amount of the psychedelic compound to the patient by intravenous infusion comprises administering:
a first dose of said psychedelic compound at a first infusion rate for a first infusion time; and
a second dose of said psychedelic compound at a second infusion rate for a second infusion time;
wherein the first and second infusion rates are different.

13. The method according to claim 12, wherein the first infusion rate is at least 20%, at least 40%, at least 60%, at least 80%, at least 100%, at least 150%, or at least 200% greater than the second infusion rate.

14. The method according to claim 12 or claim 13, wherein the first infusion rate is from about 0.05 mg/min to about 0.3 mg/min, from about 0.08 mg/min to about 0.25 mg/min, or from about 0.1 mg/min to about 0.2 mg/min of the psychedelic compound.

15. The method according to any one of claims 12 to 14, wherein the second infusion rate is from about 0.01 mg/min to about 0.15 mg/min, from about 0.03 mg/min to about 0.1 mg/min, or from about 0.04 mg/min to about 0.08 mg/min of the psychedelic compound.

16. The method according to any one of claims 12 to 15, wherein the first infusion time is from about 1 minute to about 40 minutes, from about 5 minutes to about 30 minutes, or from about 8 minutes to about 25 minutes.

17. The method according to any one of claims 12 to 16, wherein the second infusion time is from about 45 minutes to about 200 minutes, from about 60 minutes to about 150 minutes, or from about 90 minutes to about 135 minutes.

18. The method according to any one of claims 12 to 17, wherein:
the first dose is less than or equal to 50%, less than or equal to 40%, or less than or equal to 30% of the second dose.

19. The method according to any one of claims 12 to 18, wherein:
the first dose is administered before the second dose; and
administration of the second dose begins upon completion of administration of the first dose.

20. The method according to any one of the preceding claims, wherein the method comprises administering a pharmaceutical composition comprising a therapeutically effective amount of the psychedelic compound to a patient by intravenous infusion,
which pharmaceutical composition is a formulation suitable for intravenous infusion comprising the psychedelic compound and one or more pharmaceutically acceptable carriers, solvents, solubilisers, diluents, adjuvants, excipients, or vehicles.

21. The method according to claim 20, wherein the formulation comprises the psychedelic compound, at least one pharmaceutically acceptable diluent, and optionally at least one pharmaceutically acceptable solubiliser.

22. The method according to claim 21, wherein the formulation comprises the psychedelic compound at a concentration of at least about 200 mg/g.

23. The method according to any one of claims 20 to 22, wherein the formulation is a solution comprising the psychedelic compound, 2-hydroxypropyl-β-cyclodextrin, and water, and wherein the psychedelic compound is psilocin.

24. The method according to any one of claims 20 to 22, wherein the formulation is a solution comprising the psychedelic compound and water, and wherein the psychedelic compound is psilocin.

25. The method according to any one of claims 20 to 24, wherein the method further comprises producing the formulation suitable for intravenous infusion by (a) defrosting a frozen solution, (b) reconstituting a lyophilised product, (c) solubilising a powder for infusion, or (d) diluting a concentrate for infusion.

26. The method according to any one of the preceding claims, wherein the disease or condition is selected from a psychological, neurological and central nervous system disorder, and is preferably selected from depression, anxiety, death anxiety, demoralization, adjustment disorders, hopelessness, suicidal ideation and desire for hastened death.

27. The method according to any one of claims 1 to 25, wherein the disease or condition is selected from cocaine-related disorders, opioid-related disorders and stimulant-related disorders.

28. A psychedelic compound for use in a method of treating or preventing a disease or condition in a patient, wherein:
the psychedelic compound is psilocybin or psilocin, or a pharmaceutically acceptable salt thereof; and
the method comprises administering a therapeutically effective amount of a psychedelic compound to the patient by intravenous infusion.

29. A psychedelic compound for use according to claim 28, wherein:
administering the psychedelic compound by intravenous infusion is as further defined in any one of claims 2 to 25.

30. The psychedelic compound for use according to claim 28 or claim 29, wherein the psychedelic compound is for use in the treatment of a disease or condition selected from a psychological, neurological and central nervous system disorder, and preferably selected from depression, anxiety, death anxiety, demoralization, adjustment disorders, hopelessness, suicidal ideation, desire for hastened death.

31. The psychedelic compound for use according to claim 28 or claim 29, wherein the psychedelic compound is for use in the treatment of a disease or condition selected from cocaine-related disorders, opioid-related disorders and stimulant-related disorders.

32. Use of a psychedelic compound in the manufacture of a medicament for use in a method of treating or preventing of a disease or condition in a patient, wherein:
the psychedelic compound is psilocybin or psilocin, or a pharmaceutically acceptable salt thereof; and
the method comprises administering a therapeutically effective amount of a psychedelic compound to the patient by intravenous infusion.

33. A formulation suitable for administration by intravenous infusion comprising a psychedelic compound which is psilocin or a pharmaceutically acceptable salt thereof, wherein the formulation is a solution and the formulation comprises psilocin at a concentration of at least about 200 mg/g.

34. The formulation according to claim 33, wherein the formulation further comprises at least one pharmaceutically acceptable diluent, and optionally further comprises at least one pharmaceutically acceptable solubiliser.

35. The formulation according to claim 34, wherein the diluent is water.

36. The formulation according to claim 34 or claim 35, wherein the solubiliser is 2-hydroxypropyl-β-cyclodextrin.

37. The formulation according to any one of claims 33 to 36, wherein the formulation is a solution comprising 2-hydroxypropyl-β-cyclodextrin and water.

38. The formulation according to claim 37, wherein the formulation comprises 2-hydroxypropyl-β-cyclodextrin at a concentration of from 5 to 15% w/v, and water at a concentration of from 85 to 95% w/v.

39. The formulation according to any one of claims 33 to 36, wherein the formulation is a solution comprising water.

40. The formulation according to claim 39, wherein the formulation comprises 100% w/v water.

41. A formulation according to any one of claims 33 to 40, wherein the formulation is stable for 7 days at 2-8° C.

42. A kit comprising:
one or more infusion bags, syringes, vials and/or ampoules comprising a formulation suitable for administration by intravenous infusion, which formulation comprises a psychedelic compound which is psilocybin or psilocin, or a pharmaceutically acceptable salt thereof; and
instructions for use of the one or more infusion bags or syringes in a method as defined in any one of claims 1 to 27.

43. The kit of claim 42, wherein each formulation is as defined in any one of claims 33 to 41.

44. A device which is configured to administer a psychedelic compound to a patient by intravenous infusion, wherein:
the psychedelic compound is psilocybin or psilocin, or a pharmaceutically acceptable salt thereof; and
administering the psychedelic compound to a patient by intravenous infusion is as further defined in any one of claims 1 to 27.

45. The device of claim 44, wherein the psychedelic compound is administered to the patient as a formulation as defined in any one of claims 33 to 41.
